(19) United States
(12) Patent Application Publication
(10) Pub. No.: US 2004/0028634 A1
(43) Pub. Date: Feb. 12, 2004

(54) Skin care composition

(75) Inventor: Hidekazu Tanaka, Kobe (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/627,383

(22) Filed: Jul. 25, 2003

Related U.S. Application Data
(63) Continuation of application No. PCT/US01/02359, filed on Jan. 25, 2001.

(51) Int. Cl. A61K 7/06; A61K 7/11

(52) U.S. Cl. 424/70.12

(57) ABSTRACT

Disclosed is a skin care composition comprising: (1) from about 0.1% to about 1% of a carboxylic acidicarboxylate copolymer; (2) from about 0.5% to about 10% of a tacky skin treatment agent; (3) from about 2% to about 20% of a water soluble humectant; (4) from about 0.5% to about 5% of an emollient oil having a viscosity of less than about 50 mPa·s; (5) from about 0.05% to about 5% of a silicone component; and (6) an aqueous carrier.

### CROSS REFERENCE TO RELATED APPLICATION(s)

This application is a continuation of International Application No. PCT/US01/02359, designating the U.S., filed Jan. 25, 2001.

### TECHNICAL FIELD

The present invention relates to skin care compositions comprising a carboxylic acid/carboxylate copolymer, a tacky skin treatment agent, a water soluble humectant, an emollient oil of low viscosity, a silicone component, and an aqueous carrier, which provides improved skin feel.

### BACKGROUND

Many personal care products currently available to consumers are directed primarily to improving the health and/or physical appearance of the skin. Among these skin care products, many are directed to delaying, minimizing or even eliminating skin wrinkling and other histological changes typically associated with skin aging or environmental damage to human skin.

Skin is subject to insults by many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin. Whether extrinsic or intrinsic, these factors result in visible signs of skin aging and environmental damage such as wrinkling and other forms of roughness (including increased pore size, flaking and skin lines), and other histological changes associated with skin aging or damage. Signs of skin aging include, but are not limited to, all outward visibly and tactilely perceptible manifestations as well as any other macro or micro effects.

In order to maintain or return skin to a healthy and/or youthful state, the skin is typically treated with a moisturizing agent. Known moisturizing agents include, for example, glycerin. Increasing the level of moisturizing agent applied to the skin typically provides improved moisturization of the skin.

Popular forms of skin care products employed for skin moisturization include clear lotions, milk lotions, and essences. Such compositions typically have a high water content. Thus, consumers expect such high water content compositions to have a non-greasy feeling. This is particularly true for clear lotions which have a transparent or translucent appearance. Unfortunately, as the percentage of moisturizing agent is increased in such compositions, these compositions tend to impart a greasy feeling and result in poor distribution/spreading of the moisturizing agent to the skin.

Aqueous compositions employing specific silicone components in addition to water-soluble humectants are known, such as in U.S. Pat. No. 5,420,118. While the compositions disclosed in this reference are capable of delivering water-soluble humectants for moisturization, further improvement is desired for reducing tacky and greasy feeling to the skin. This is particularly true when skin treatment agents which imply tacky feeling, such as panthenol and niacinamide, are incorporated in an aqueous composition.

Based on the foregoing, there is a need for an aqueous skin care composition containing tacky skin treatment agents which can improve reduction of tacky/greasing feeling to the skin.

None of the existing art provides all of the advantages and benefits of the present invention.

### SUMMARY

The present invention is directed to a skin care composition comprising:

(1) from about 0.1% to about 1% of a carboxylic acid/carboxylate copolymer;

(2) from about 0.5% to about 10% of a tacky skin treatment agent;

(3) from about 2% to about 20% of a water soluble humectant;

(4) from about 0.5% to about 5% of an emollient oil having a viscosity of less than about 50 mPa·s;

(5) from about 0.05% to about 5% of a silicone component; and

(6) an aqueous carrier.

These and other features, aspects, and advantages of the present invention will become evident to those skilled in the art from a reading of the present disclosure.

### DETAILED DESCRIPTION

While the specification concludes with claims which particularly point out and distinctly claim the invention, it is believed the present invention will be better understood from the following description.

All cited references are incorporated herein by reference in their entireties. Citation of any reference is not an admission regarding any determination as to its availability as prior art to the claimed invention.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

The aspects and embodiments of the present invention set forth in this document have many advantages. For example, the skin care compositions of the present invention provide texture improvement of the skin due to the tacky treatment agents being effectively delivered to the skin and providing imbibition of stratum corneum cells without leaving a tacky and/or greasing feeling on the skin upon use. The skin care compositions of the present invention further provide a transparent or translucent appearance.

Carboxylic Acid/Carboxylate Copolymer

The compositions of the present invention comprise from about 0.1% to about 1%, preferably from about 0.1% to about 0.5% of a carboxylic acid/carboxylate copolymer. The carboxylic acid/carboxylate copolymer is capable of dispersing and stabilizing the emollient oils and silicone components in an aqueous environment, so that such components do not separate out. In addition, the carboxylic acid/carboxylate copolymer keeps the composition relatively transparent and at a suitable viscosity without making the composition tacky or greasy upon use. The carboxylic acid/carboxylate copolymer is particularly advantageous for providing a skin lotion composition having a viscosity of no more than about 4000 mPa·s.

The carboxylic acid/carboxylate copolymers herein are hydrophobically-modified cross-linked coplymers of carboxylic acid and alkyl carboxylate, and have an amphiphilic property. These carboxylic acid/carboxylate copolymers are obtained by copolymerizing 1) a carboxylic acid monomer such as acrylic acid, methacrylic acid, maleic acid, maleic anhydride, itaconic acid, fumaric acid, crotonic acid, or α-chloroacrylic acid, 2) a carboxylic ester having an alkyl chain of from 1 to about 30 carbons, and preferably 3) a crosslinking agent of the following formula:

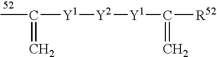

wherein R⁵² is a hydrogen or an alkyl group having from about 1 to about 30 carbons; Y¹, indepedently, is oxygen, CH₂O, COO, OCO,

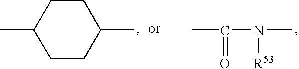

wherein R⁵³ is a hydrogen or an alkyl group having from about 1 to about 30 carbons; and Y² is selected from (CH₂)_{m″}, (CH₂CH₂O)_{m″}, or (CH₂CH₂CH₂O)_{m″} wherein m″ is an integer of from 1 to about 30. It is believed that, because of the alkyl group contained in the copolymer, the carboxylic acid/carboxylate copolymers do not make the composition undesirably sticky.

Suitable carboxylic acid/carboxylate copolymers herein are acrylic acid/alkyl acrylate copolymers having the following formula:

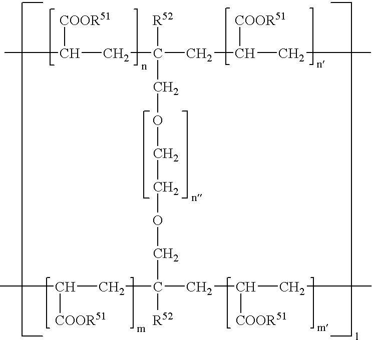

wherein R⁵¹, independently, is a hydrogen or an alkyl of 1 to 30 carbons wherein at least one of R⁵¹ is a hydrogen, R⁵² is as defined above, n, n′, m and m′ are integers in which n+n′+m+m′ is from about 40 to about 100, n″ is an integer of from 1 to about 30, and l is defined so that the copolymer has a molecular weight of about 500,000 to about 3,000,000.

Commercially available carboxylic acid/carboxylate copolymers useful herein include: CTFA name Acrylates/C10-30 Alkyl Acrylate Crosspolymer having tradenames Pemulene TR-1, Pemulene TR-2, Carbopol 1342, Carbopol 1382, and Carbopol ETD 2020, all available from B. F. Goodrich Company.

Neutralizing agents may be included to neutralize the carboxylic acid/carboxylate copolymers herein. Nonlimiting examples of such neutralizing agents include sodium hydroxide, potssium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

Emollient Oil

The composition of the present invention comprises from about 0.5% to about 5%, preferably from about 1% to about 3% of an emollient oil. The emollient oil useful herein are those which have a viscosity of less than about 50 mPa·s, have a melting point of not more than about 25° C., and provide emollient benefit to the skin. The emollient oils useful herein may be volatile or nonvolatile, and include esters and hydrocarbons. It has been surprisingly found that, by the use of these specific low viscosity oils, the tacky and greasy feel to the skin can be alleviated.

Emollient oils useful herein are esters, particularly esters having branched alkyl and alkenyl groups, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, isostearyl myristate, octyl palmitate, caprylic/capric acid triglyceride, glyceryl tri-2-ethylhexanoate, neopentyl glycol di(2-ethyl hexanoate), neopentyl glycol dicaprate, diisopropyl dimerate, glycerol trioctanate, glycerol triisopalmitate, isopropyl myristate, octyidodecyl lactate, and mixtures thereof. Commercially available oils include, for example, isononyl isononanoate with tradenames Salacos 99 available from Nisshin Oil Mills, or Lanol 99 available from Seppic; tridecyl isononanoate with tradename Crodamol TN available from Croda, and Hexalan available from Nisshin Seiyu.

Emollient oils also useful herein are the various grades and types of hydrocarbons. Mineral oils are liquid mixtures of hydrocarbons that are obtained from petroleum. Specific examples of suitable hydrocarbons include paraffin oil, mineral oil, squalane, dodecane, isododecane, hexadecane, isohexadecane, eicosene, isoeicosene, tridecane, tetradecane, and mixtures thereof. Commercially available hydrocarbons useful herein include isododecane, isohexadeance, and isoeicosene with tradenames PERMETHYL 99A, PERMETHYL 101A, and PERMETHYL 1082, available from Presperse (South Plainfield N.J., USA), mineral oil with tradename BENOL available from Witco, isoparaffin with tradename ISOPAR from Exxon Chemical Co. (Houston Tex., USA.)

Silicone Component

The compositions of the present invention comprise from about 0.05% to about 5%, preferably from about 0.5% to about 3% of a silicone component. The silicone components useful herein include volatile or nonvolatile insoluble silicone conditioning agents. By insoluble what is meant is that the silicone forms a separate, discontinuous phase from the carrier, such as in the form of an emulsion or a suspension of droplets of the silicone. The silicone components herein may be made by any suitable method known in the art, including emulsion polymerization. The silicone components may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof. Silicone components of high molecular weight may be made by emulsion polymerization.

Silicone components useful herein include polyalkyl polyaryl siloxanes, silicone resins, amino-substituted siloxanes, and mixtures thereof. The silicone component is preferably selected from the group consisting of polyalkyl polyaryl siloxanes, silicone resins, and mixtures thereof, and more preferably from one or more polyalkyl polyaryl siloxanes.

Polyalkyl polyaryl siloxanes useful here in include those with the following structure (I)

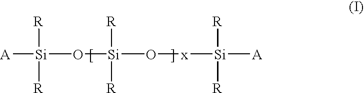

wherein R is alkyl or aryl, and x is an integer from about 7 to about 8,000. “A” represents groups which block the ends of the silicone chains. The alkyl or aryl groups substituted on the siloxane chain (R) or at the ends of the siloxane chains (A) can have any structure as long as the resulting silicone remains fluid at room temperature, is dispersible, is neither irritating, toxic nor otherwise harmful when applied to the skin, is compatible with the other components of the composition, is chemically stable under normal use and storage conditions, and is capable of being deposited on and conditions the skin. Suitable A groups include hydroxy, methyl, methoxy, ethoxy, propoxy, and aryloxy. The two R groups on the silicon atom may represent the same group or different groups. Preferably, the two R groups represent the same group. Suitable R groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their ViscasilR and SF 96 series, and from Dow Corning in their Dow Corning 200 series. Polymethylphenylsiloxanes, for example, from the General Electric Company as SF 1075 methyl phenyl fluid or from Dow Corning as 556 Cosmetic Grade Fluid, are useful herein.

Another polyalkyl polyaryl siloxane that can be especially useful is a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. Silicone gums are described by Petrarch, and others including U.S. Pat. No. 4,152,416, to Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. All of these described references are incorporated herein by reference in their entirety. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof.

Also useful are silicone resins, which are highly crosslinked polymeric siloxane systems. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art. Without being bound by theory, it is believed that the silicone resins can enhance deposition of other silicone components on the skin.

The method of manufacturing these silicone components, can be found in Encyclopedia of Polymer Science and Engineering, Volume 15, Second Edition, pp. 204-308, John Wiley & Sons, Inc., 1989.

In one preferred embodiment, the silicone component is a mixture of high viscosity silicone compounds and silicone based carriers.

High viscosity silicone compounds herein include those having a molecular weight of from about 200,000 to about 540,000 selected from those mentioned above, preferably selected from the group consisting of dimethiconol, fluorosilicone dimethicone, and mixtures thereof, more preferably essentially dimethiconol. Particularly preferred dimethiconols are those having dimethylpolysiloxane repeating units, and terminated with hydroxy groups, wherein the dimethylsiloxane portion is made of from about 2700 to about 4500 repeating units.

Silicone based carriers include those having a viscosity of from about 0.65 mPa·s to about 100 mPa·s selected from cyclomethicones and dimethicones having lower repeating units.

Commercially available silicone components which are useful herein include Dimethicone with tradename DC345 available from Dow Corning Corporation, Dimethicone gum solutions with tradenames SE 30, SE 33, SE 54 and SE 76 available from General Electric, Dimethiconol with tradenames DCQ2-1403 and DCQ2-1401 available from Dow Corning Corporation, and emulsion polymerized Dimethiconol available from Toshiba Silicone as described in GB application 2,303,857.

Tacky Skin Treatment Agent

The composition of the present invention comprise from about 0.5% to about 10%, preferably from about 1% to about 5% of a tacky skin treatment agent. Skin treatment agents useful herein are those which help repair and replenish the natural moisture barrier function of the epidermis, thereby providing skin benefits such as texture improvement. It is generally known that, while such agents provide useful benefits to the skin when used chronically, they also tend to provide negative skin feel upon use when applied by itself.

Tacky skin treatment agents useful herein are niacinamide, nicotinic acid and its esters, nicotinyl alcohol, panthenol, panthenyl ethyl ether, n-acetyl cysteine, n-acetyl-L-serine, phosphodiesterase inhibitors, trimethyl glycine, tocopheryl nicotinate, and vitamin D3 and analogues or derivatives, and mixtures thereof. Niacinamide is particularly preferred in that, when used in a pharmaceutically effective amount, is capable of reducing or alleviating the intensity of chronical spots. Niacinamide is suitably incorporated in the composition by first dissolving in water. Panthenol is also particularly preferred in that, when used in an amount of at least about 1%, it provides texture improvement benefits. Niacinamide and panthenol are commercially available, for example, by Roche.

Water Soluble Humectant

The composition of the present invention comprise from about 2% to about 20%, preferably from about 5% to about 15% of a water soluble humectant. In the compositions of the present invention, water soluble humectants are comprised in addition to the tacky skin treatment agents described above.

Water soluble humectants useful herein include polyhydric alcohols such as sorbitol, propylene glycol, butylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, and mixtures thereof.

Water soluble humectants useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

Commercially available humectants herein include: glycerin with tradenames STAR and SUPEROL available from The Procter & Gamble Company, CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; sorbitol with tradenames LIPONIC series available from Lipo, SORBO, ALEX, A-625, and A-641 available from ICI, and UNISWEET 70, UNISWEET CONC available from UPI; dipropylene glycol with the same tradename available from BASF; diglycerin with tradename DIGLYCEROL available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate with tradenames ACTIMOIST available from Active Organics, AVIAN SODIUM HYALURONATE series available from Intergen, HYALURONIC ACID Na available from Ichimaru Pharcos; sodium adenosin phophate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; and polyethylene glycols with the tradename CARBOWAX series available from Union Carbide.

Aqueous Carrier

The compositions of the present invention comprise an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, the aqueous carrier is substantially water. Deionized water is preferably used. Water from natural sources including mineral cations can also be used, depending on the desired characteristic of the product.

The pH of the present composition is preferably from about 4 to about 8, more preferably from about 5 to about 7. The suitable tacky skin treatment agents are particularly efficient in such pH range. Buffers and other pH adjusting agents can be included to achieve the desirable pH.

Additional Viscosity Modifier

The compositions of the present invention may further comprise an additional viscosity modifier. The additional viscosity modifiers herein are water soluble or water miscible polymers, have the ability to increase the viscosity of the composition, and are compatible with the carboxylic acid/carboxylate copolymers. The additional viscosity modifier is selected so that the composition of the present composition has a suitable viscosity, preferably from about 100 mPa·s to about 4000 mPa·s, more preferably from about 100 mPa·s to about 3000 mPa·s, still preferably from about 100 mPa·s to about 2000 mPa·s. If such a viscosity is achieved without the additional viscosity modifier, the additional viscosity modifier may not be necessary. The viscosity herein can be suitably measured by Brookfield LV at 20rpm at 25° C. using either spindle #4, 5, 6 or 7 depending on the viscosity and the characteristic of the composition.

Additional viscosity modifiers useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Polyalkylenn glycols having a molecular weight of more than about 1000 are useful herein. Useful are those having the following general formula:

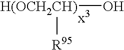

wherein R⁹⁵ is selected from the group consisting of H, methyl, and mixtures thereof. When R⁹⁵ is H, these materials are polymers of ethylene oxide, which are also known as polyethylene oxides, polyoxyethylenes, and polyethylene glycols. When R⁹⁵ is methyl, these materials are polymers of propylene oxide, which are also known as polypropylene oxides, polyoxypropylenes, and polypropylene glycols. When R⁹⁵ is methyl, it is also understood that various positional isomers of the resulting polymers can exist. In the above structure, x3 has an average value of from about 1500 to about 25,000, preferably from about 2500 to about 20,000, and more preferably from about 3500 to about 15,000.

Other useful polymers include the polypropylene glycols and mixed polyethylene-polypropylene glycols, or polyoxyethylene-polyoxypropylene copolymer polymers. Polyethylene glycol polymers useful herein are PEG-2M wherein R⁹⁵ equals H and x3 has an average value of about 2,000 (PEG-2M is also known as Polyox WSR® N-10, which is available from Union Carbide and as PEG-2,000); PEG-5M wherein R⁹⁵ equals H and x3 has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R⁹⁵ equals H and x3 has an average value of about 7,000 (PEG-7M is also known as Polyox WSR® N-750 available from Union Carbide); PEG-9M wherein R⁹⁵ equals H and x3 has an average value of about 9,000 (PEG 9-M is also known as Polyox WSR® N-3333 available from Union Carbide); and PEG-14 M wherein R⁹⁵ equals H and x3 has an average value of about 14,000 (PEG-14M is also known as Polyox WSR® N-3000 available from Union Carbide).

Commercially available additional viscosity modifiers highly useful herein include xanthan gum with tradename Keltrol series available from Kelco, Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Herculus, ethylene oxide and/or propylene oxide based polymers with tradenames CARBOWAX PEGs, POLYOX WASRs, and UCON FLUIDS, all supplied by Amerchol.

Other Components

Other components which can be formulated into the compositions of the present invention are; preservatives such as benzyl alcohol, methyl paraben, propyl paraben, imidazolidinyl area, and EDTA and its salts, perfumes, ultraviolet and infrared screening and absorbing agents, yeast fermented filtrates, and others.

Compositions

The composition of the present invention is suitably for topical use on human body skin, particularly suitable for facial skin. The use of the present composition provides texture improvement of the skin due to the tacky treatment agents being effectively delivered to the skin and providing imbibition of stratum corneum cells without leaving a tacky and/or greasing feeling on the skin upon use. The skin care compositions of the present invention further provide a transparent or translucent appearance.

The composition of the present invention may also be applied on other areas of the body, the hair, or scalp for moisturization of such areas.

The composition of the present invention may be made by methods known in the art. In a suitable process, the composition is made by the steps of:

(a) adding the carboxylic acid/carboxylate copolymer into at least a portion of water and mixing until homogeneous;

(b) adding the silicone component and the emollient oil to the product of step (a) and mixing until homogeneous; and

(c) adding the remainder of the composition to the product of step (b).

In step (a), the carboxylic acid/carboxylate copolymer is dispersed in water to make the base carrier of the composition. All or portion of the water may be used. Appropriate mixing is provided to make the dispersion homogeneous. Heat may be added to aid the dispersion, preferably up to about 70° C. In step (b), components of hydrophobic nature, i.e. the silicone component and emollient oil, is included in the product of step (a), and mixed until homogeneous. The rotation of mixing should be adjusted so that the obtained mixture is homogenous, yet not overly agitated to affect phase stability. Preferably, mixing is made at a rotation speed of no more than about 500 pm. Neutralizing agents for the carboxylic acid/carboxylate copolymer are added after step (b), when used. Finally the remaining components, including the tacky skin treatment agent and water soluble humectants, and remainder of water and additional viscosity modifier, if present, are added and mixed. Typically, heating is not necessary in steps (b) and (c). This is particularly true when components added in these steps are freely fluid in nature. In one preferred embodiment, the components added in steps (b) and (c) are freely fluid, and steps (b) and (c) are carried out at room temperature.

The obtained composition preferably has a viscosity of from about 100 mPa·s to about 4000 mPa·s, more preferably from about 100 mPa·s to about 3000 mPa·s, still preferably from about 100 mPa·s to about 2000 mPa·s. In one preferred embodiment, such relatively low viscosity composition is suitable for use as a skin lotion on the facial skin.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

Compositions

| | Ex. 1 | Ex. 2 | Ex. 3 | Ex. 4 | Ex. 5 | Ex. 6 |
|---|---|---|---|---|---|---|
| Acrylic acid alkyl acrylate | | | | | | 0.2 |
| copolymer 1 *1 | | | | | | |
| Acrylic acid alkyl acrylate | 0.24 | 0.24 | 0.24 | 0.24 | 0.2 | |
| copolymer 2 *2 | | | | | | |
| Aminomethyl propanol | 0.12 | 0.12 | 0.14 | 0.12 | 0.15 | 0.1 |
| Panthenol *3 | 1.0 | 1.0 | 1.0 | 1.0 | 2.0 | 1.0 |
| Niacinamide *4 | 2.0 | 2.0 | 2.0 | 2.0 | 2.0 | 2.0 |
| Glycerin | 6.5 | 6.5 | 6.5 | 6.5 | 5.0 | 5.0 |
| 1,3-butytene glycol | 0.08 | 0.08 | 0.08 | 0.08 | 8.0 | 8.0 |
| Sodium Hyaluronate *5 | | | | | 0.02 | 0.02 |
| lsohexadecane *6 | 2.0 | | | 2.0 | 2.5 | 2.5 |
| lsododecane *7 | | 2.0 | | | | |
| Isononyl isononanoate *8 | | | 2.0 | | | |
| Dimethicone/Dimethiconol *9 | 1.5 | 1.5 | 1.5 | | 1.0 | |
| Cyclomethicone/Di- | | | | 1.5 | | 1.0 |
| methiconol *10 | | | | | | |
| Xanthan gum *11 | 0.04 | 0.04 | 0.04 | 0.04 | | |
| Carbomer *12 | | | | | 0.2 | |
| Methyl Paraben | 0.15 | 0.15 | 0.15 | 0.15 | 0.15 | 0.15 |
| Benzyl alcohol | 0.25 | 0.25 | 0.25 | 0.25 | 0.25 | 0.25 |
| Sodium benzoate | 0.05 | 0.05 | 0.05 | 0.05 | 0.05 | 0.05 |
| EDTA-2Na | 0.1 | 0.1 | 0.1 | 0.1 | 0.1 | 0.1 |

| | |
|---|---|
| Deionized Water | q.s. to 100% |

| | Ex. 7 | Ex. 8 | Ex. 9 | Ex. 10 |
|---|---|---|---|---|
| Acrylic acid alkyl acrylate copolymer 1 *1 | | | 0.1 | 0.1 |
| Acrylic acid alkyl acrylate copolymer 2 *2 | 0.4 | 0.4 | 0.3 | 0.2 |
| Aminomethyl propanol | 0.15 | 0.15 | 0.15 | 0.2 |
| Panthenol *3 | | 1.0 | 2.0 | 1.0 |
| Panthenyl ethyl ether *13 | 2.0 | 1.0 | | 2.0 |
| Niacinamide *4 | 2.0 | 2.0 | 2.0 | 3.0 |
| Glycerin | 5.0 | 5.0 | 5.0 | 8.0 |
| 1,3-butylene glycol | 5.0 | 5.0 | 5.0 | 2.0 |
| Isohexadecane *6 | 2.0 | 2.0 | | |
| Isododecane *7 | | | 3.0 | |
| Iosononyl isononanoste *8 | | | | 2.0 |
| Dimethicone/Dimethiconol *9 | 1.5 | | 1.0 | |
| Cyclomethicone/Dimethiconol *10 | | 2.0 | | 2.0 |
| Carbomer *12 | | | | 0.2 |
| Methyl Paraben | 0.15 | 0.15 | 0.15 | 0.15 |
| Benzyl alcohol | 0.25 | 0.25 | 0.25 | 0.25 |
| Sodium benzoate | 0.05 | 0.05 | 0.05 | 0.05 |
| EDTA-2Na | 0.1 | 0.1 | 0.1 | 0.1 |
| Ascosporogeneous Yeast Fermented | 30 | | | |
| Filtrate *14 | | | | |

| | |
|---|---|
| Deionized Water | q.s. to 100% |

Definitions of Components
*1 Acrylic acid alkyl acrylate copolymer 1: PEMULEN TR-1 available from B. F. Goodrich
*2 Acrylic acid alkyl acrylate copolymer 2: PEMULEN TR-2 available from B. F. Goodrich
*3 Panthenol: available from Roche
*4 Niacinamide: available from Roche
*5 Sodium Hyaluronate: available from Chisso Corp.
*6 Isohexadecane: Permethyl 101A available from Presperse
*7 Isododecane: Permethyl 99A available from Presperse
*8 Isononyl isononanoate: Salacos 99 available from Nisshin Oil Mills, or Lanol 99 available from Seppic
*9 Dimethicone/Dimethiconol: DCQ2-1403 available from Dow Corning
*10 Cyclomethicone/Dimethiconol: DCQ2-1401 available from Dow Corning
*11 Xanthan gum: Keltrol T available from Kelco
*12 Carbomer: Carbopol 981 available from B. F. Goodrich
*13 Panthenyl ethyl ether: Ethyl panthenol available from Roche
*14 Yeast Ferment Filtrate: SKII Pitera available from Kashiwayama

Method of Preparation

The polymeric materials such as the carboxylic acid/alkyl carboxylate copolymer, are dispersed in a portion of water at room temperature, mixed at a rotation speed controlled to no more than 5000 rpm, of by vigorous agitation, and heated to about 70° C. until homogenous. A triblender can be used if necessary to disperse the polymeric materials. To this mixture, the silicone component and the emollient oil are added. The neutralizing agent, if present, is added to the mixture. After neutralizing, a water solution of the remaining components including tacky skin treatment agents, water soluble humectants, additional viscosity modifier, if present, and other components, if present, are added to the mixture, and then cooled to below 40° C.

Examples 1 through 10 are particularly useful for providing clear lotions for use on the facial skin. When used on the facial skin, the compositions of Examples 1 through 10 provide moisturizing benefit to the skin without leaving a tacky and/or greasy feel to the skin.

It is understood that the examples and embodiments described herein are for illustrative purposes only and that various modifications or changes in light thereof will be suggested to one skilled in the art without departing from its spirit and scope.

What is claimed is:

1. A skin care composition comprising:
(1) from about 0.1% to about 1% of a carboxylic acid/carboxylate copolymer;
(2) from about 0.5% to about 10% of a tacky skin treatment agent;
(3) from about 2% to about 20% of a water soluble humectant;
(4) from about 0.5% to about 5% of an emollient oil having a viscosity of less than about 50 mPa·s;
(5) from about 0.05% to about 5% of a silicone component; and
(6) an aqueous carrier.

2. The skin care composition according to claim 1 wherein the composition has a viscosity of from about 100 mPa·s to about 400 mPa·s.

3. The skin care composition according to claim 1 wherein the tacky skin treatment agent is comprised at a level of at least 1% and is selected from the group consisting of panthenol, niacinamide, and mixtures thereof.

4. The skin care composition according to claim 1 wherein the emollient oil is selected from hydrocarbons.

5. The skin care composition according to claim 1 wherein the silicone component is a mixture of high viscosity silicone compounds and silicone based carriers.

6. The skin care composition according to claim 5 wherein the high viscosity silicone compound is dimethiconol.

7. The skin care composition according to claim 1 further comprising an additional viscosity modifier.

8. A method of improving skin texture comprising the step of applying the composition of claim 3 to the skin.

9. A method of preparing the composition of claim 1 comprising the steps of:
(a) adding the carboxylic acid/carboxylate copolymer into at least a portion of water and mixing until homogeneous;
(b) adding the silicone component and the emollient oil to the product of step (a) and mixing until homogeneous; and
(c) adding the remainder of the composition to the product of step (b).

10. The method according to claim 9 wherein the mixing at step (a) is provided at a rotation speed of no more than about 5000 rpm.
